(19) United States
(12) Patent Application Publication
(10) Pub. No.: US 2006/0217775 A1
(43) Pub. Date: Sep. 28, 2006

(54) Self test system for a medical device

(75) Inventors: Desmond Bryan Mills, Gloucestershire (GB); Kevin Herbert, Gloucestershire (GB)

Correspondence Address: YOUNG & THOMPSON, 745 SOUTH 23RD STREET, 2ND FLOOR, ARLINGTON, VA, 22202, US

(73) Assignee: IKA Medical Products LLP, Cheltenham (GB)

(21) Appl. No.: 10/559,940

(22) PCT Filed: Jun. 14, 2004
(86) PCT No.: PCT/GB04/02522

§ 371 Date: Mar. 17, 2006

(30) Foreign Application Priority Data
Jun. 14, 2003 (GB) 0313815.3

(51) Int. Cl. A61N 1/00

(52) U.S. Cl. 607/27; 128/897

(57) ABSTRACT

A self test system for a medical device the medical device being arranged to send information concerning components of the medical device to an indicator which can show the status of the components when tested, characterised in that the self test system includes one or more self test units which can self test one or more individual components of the medical device, the self test being activated independently of operation of the medical device and not by a signal from a processor associated with the medical device.

The invention relates to a self test system for a medical device in which the device includes a summator to collate information about components of the device. In particular, the invention is directed to an AED (automatic external defibrillator) having a summator to collate information about the individual components making up the AED so that the person being treated can be treated as safely and efficiently as possible.

Known AED's include self test systems whereby a self test signal is generated by a processor associated with the AED and the sub-components of the AED are caused to carry out a self test routine. The results of the self tests are then individually fed to a processor, which initiated the tests. Once processed, the data can be displayed to the device operator using an indicator panel, such as a LCD.

In these known systems, there may be a delay in operation because a signal has to be sent from a central processing unit to the components to induce them to carry out a self test. The test results then are sent to a processor prior to the results being displayed to an operator by an indicator. Further, because the components do not include their own self test facility, independently of any other operation of the testing of the medical device, all components are tested, rather then there being the facility for selected components to be tested, which increases costs due to the fact that time is expended carrying out the test that may not be necessary. There is no facility whereby activated self test of components is carried out to see if they are at least to some degree in an operable condition so in future tests only these components which were found to possibly not be in an operable condition are tested further to see if they are in a condition to be used with the medical device. The present invention aims to overcome problems associated with the prior art.

According to a first aspect of the invention there is provided a self test system for a medical device said medical device being arranged to send information concerning components of said medical device to an indicator which can show the status of the components when tested, characterised in that the self test system comprises one or more self test units which can self test one or more individual components of the medical device, the self test being activated independently of operation of the medical device and not by a signal from a processor associated with said medical device. It is preferred that the processor is not a centralized processor i.e, not a processor that is a controlling unit for the test system or the medical device.

Preferably the self test system includes a summator which receives data from the one or more self test units about said components, the summator storing said data so that it can be transmitted to an indicator either directly or via a processor which can access said data.

According to a further aspect of the invention there is provided a self test system for a medical device said medical device being arranged to transmit information concerning components of said medical device to an indicator which can show the status of the components when tested, characterised in that the self test system comprises one or more self test units which can self test one or more individual components of the medical device and a summator which receives data from the one or more self test units about said components, the summator storing said data so that it can be transmitted to an indicator either directly or via a processor which can access said data.

It is envisaged that components such as, for example, the pads for placing on a patient's chest, the circuitry associated with the pads or within the medical device, a modem associated with the medical device or a battery may be tested by the self test units. Preferably, the components each have respective dedicated self test units. However, it is envisaged that a single self test unit may test more than one component. The self test unit may have its own power source to carry out a test. With the results being stored by the summator. Once the main power for the medical device is activated, results from the summator are processed by a processing unit. Alternatively, the results can be sent directly from the summator to indicator display.

Ideally, the summator stores the data received by means of one or more latches. Typically summators latch in banks of 5 with the number of latches being equal the number of components that are tested. By latching the data, then upon enquiry by a processor, or other self test monitoring device, the last self test results latched can be retrieved as easily and efficiently as possible. Furthermore, the use of latching means that synchronous self testing is not needed and each component can have a different self test rate. This is desirable where different components have different shelf lives. An example is the case of pads that are placed on a patient's chest to monitor the heart or deliver charge, such pads may have to be tested every six hours in case they have been damaged as any damage would reduce efficiency of a medical device operating. However, circuitry, which is less prone to wear and tear, may only need to be tested once a month for example. Ideally testing should be carried out only when necessary as reduced testing avoids unnecessary utilisation of charge; retaining charge contributes to the medical device being able to operate as efficiently as possible for as long as possible. Preferably, the self test rates for each component of the self test system may be independently selectable.

It is preferred that the summator is an independent module forming part of the medical device. However, it may be provided as an external module that can be linked to the medical device when required using an adapter Ideally the summator is of the counter/adder type. However, a summator, which is of the subtractor type, could be used to subtract data values from a total to give a desired result. Further, the summator may be provided as a dedicated micro-controller, which can be a sub component of a processor. The processor only collects the data and does not send or generate a signal to the components.

Preferably, the summator has only one data input link, which receives data collected from all the self test units. However, other various arrangements may be provided for the input/output for the summator. It may be that the summator in addition to having only one input has only one output to the processor or data indicator. Alternatively, there may be separate input links from the self test units to the summator but only one output. In a further arrangement, there may be multiple input and output links to and from the summator.

It is envisaged that the input/output links are data-buses within the circuitry of the device. A databus sends or receives one piece of information at any one time. By separating out the data-buses, which data-bus being arranged to transmit data concerning one component for example, it becomes possible to send more data at any one time (parallel processing).

Preferably, when components are tested, rather than the self test units providing results in the form of discrete pulses, the pulses may be combined to give a signal at a particular level, for example a voltage level, reflecting the number of pulses received and in practice a number of pulses would form a signal. When using the combined signal, the input to the summator is kept active for an amount of time equal to the number of pulses that would normally be delivered by the unit. Using a combined signal rather than pulses allows for a more rapid-transfer rate of data to the summator, as it avoids using high and low pulse levels.

It is envisaged that each self test unit provides data about a component using a unique self test result identity so that multiple components can share the same data line without conflict. In such a situation, the identities are expressed by outputting a number of pulses in response to a self test pass to provide a unique identity number for a component. Typically, the unique identity number is chosen to have a value of x², for example 1,4,9, or to be a prime number.

It is envisaged that for a particular medical device, the unique identity number is given an upper figure. In the case of an AED, preferably this is 31. It is envisaged that a range of figures can be used for medical devices but for this particular device the battery measures its charge remaining as 30 discrete levels and outputs this along the same data-bus as 1-31 pulses (1 being zero) As the pulses are recorded in defined numerical combinations, identity numbers for components, such as a battery will be in the range of 1-31 (1 indicates a zero charge and 31 indicating a full charge). By giving one component, such as the battery, unique identity numbers, other components will not have identity numbers that conflict with those of the battery. For example, other components may have the following unique identity numbers:

| | |
|---|---|
| Electrode condition | 32² = 1024 pulses |
| Battery on charge | 33² = 1089 pulses |
| System condition | 34² = 1156 pulses |
| Modem condition | 35² = 1225 pulses |

Although the pulses may be used to indicate a positive condition for a component, they may also be used to indicate failure of components or the self test system itself.

Although the components may be self tested individually, at a rate determined for that individual component, components may also self test in response to an impulse from another component, thereby providing cascading testing. This has particular advantages where the activity of one component is dependent upon the condition of another component.

Particular components may perform specialised tests, for example if a medical device has a digital signal processor (DSP), high voltage circuit or a battery microcontroller. It is also envisaged that a test can be made to see if a component is present or not.

Preferably, in the case of a DSP the test may be induced in response to an input from the summator. When the signal is received and the test carried out, the summator is updated as to the results received from the test on the DSP. It is also envisaged that the DSP can also be prompted to self test in response to a signal from a test button activated by an operator, or alternatively upon receiving a signal from an external signal generator such as a base charger for the medical device. In a preferred arrangement, the base charger is connected to a server and the signal prompting self testing is generated by the DSP calling up the server. Ideally, the dial up will be only in response to a signal from the base station micro-controller. By using the base station to control dial up, if there were a fault with the DSP this would not stop dial up to the server.

The high voltage-circuit test may be activated as a result of tests on the pads for delivery of charge to a patient. Tests on the pads can be carried out over a defined period, for example over seven days, and the results of the tests are collated. Activation of a high voltage self test occurs when the pads have been initially connected to the device or have been tested for that defined time, or when a particular result is given for the pads in that time period. For example, if a fault in the pads is detected, a check test may be activated. A check is also made as to whether there has been a mains input signal, for example by a battery charger circuit during the seven days and if so, then the high voltage test is allowed to be carried out. By having an initial test of signal input, this ensures that the high voltage test is only carried out when the circuit has adequate charge to provide a meaningful test i.e. when the pads are connected and the device is on charge..

For a low voltage test, the tests for the pads are again collated and a low voltage self test occurs after a defined time period, which may be the same period as for the high voltage test or a different period. Where the device has a rechargeable power source a check is again made to see if there has been a mains input signal from the battery charger circuit during this time and if not, a low voltage test is carried out. This ensures that testing is limited to using a lower level of power, so that the battery power is not depleted by using a high energy test.

Further, activation of testing of the circuit may occur when the medical device is placed on charge. This test however may be over-ridden if the medical device is being charged in readiness for actual use or if another test such as testing of a server via a phone line is being carried out. This is because testing of the circuit can involve high levels of energy, and the level of charge is important for correct operation of the medical device, so ideally, the test should be carried out without interference from other test parameters.

It is envisaged that other tests may trigger testing of the circuit. A signal from a server that is transmitted either directly for server or modem or via a base charger unit to DSP may be used to prompt a voltage self test. Alternatively, manual activation of the test by a control button may occur. If there has been no test of the circuit for a defined period, then a high voltage test will be carried out. If subsequent test are requested within a defined period following the initial high voltage test, only low power self tests will occur to prevent depletion of the battery due to over use or misuse.

In a further embodiment, a battery micro-controller self test may be activated. In response to an activation signal, the battery micro-controller transmits a pulse train equivalent to the number of charges remaining (1-31). The battery monitors its overall charge in steps of 30. If no charges are remaining the pulse train is set to one pulse so that the battery always emits a signal except in the event of a battery micro-controller failure. When the battery is initially inserted it should recognise the drain of power by the AED and substantially immediately send this output pulse to verify its status to the user to ensure a depleted or damaged battery is not inserted without the user being, aware of this.

It is further envisaged that a test can be made to check whether a component is present or not, or whether a test for a component has failed. For example, if pads for delivering charge are not connected to the medical device, there should be an indication to the person carrying out the test that the pads are not present. It is envisaged that a test may be time monitored. Preferably, this is by using a clock algorithm, where if a self test unit has not reported a result for a component within a given time frame, or in the same time frame as its last report in, this fact is flagged and a signal is sent to the indicator to show a fail for the test. In a preferred arrangement, the clock itself can be tested. In this situation, the output times for the last set of 10-samples are divided by the output times for a previous 10-samples. If there is a variance in the results this would be indicated to the operator of the medical device on the indicator, that there is a system fault. Preferably, the sum of ten time samples used for the calculation.

In an alternative arrangement, rather than using a clock, the number of executions performed by a micro-controller may be used as a method of monitoring the test. If no result is for a test, this indicates that a test could not be completed and this no result will be shown by the indicator as either a fault with the micro-controller or with a component but in any event, the operator will be drawn to check the medical device to ensure it is operating properly.

In a preferred arrangement, the medical device, for example an AED, should automatically dial up a server when placed on the base station, although it may also be manually operated to effect dial up. By having automatic dial up data can be uploaded to the server to provide information about the device in the quickest possible time. Further, such a system allows the server to track movements of the device, for example when it is moved from station to station and also, when the device is replaced back in its base station, the server is informed of this which reduces the risk of an expensive piece of medical equipment being lost or poorly maintained. Ideally, the device conducts a self test when replaced in the base station so that information about the device's condition is known, which assists in preventing not only failure of the device as a whole but also gives a prediction of whether component failure is likely.

Ideally, the medical device should self test when placed on charge when in transit so that when it reaches its destination, where it could remain for some time, it will be in a charged condition ready for use. Also, during transit is a good time for self testing without compromising the rescue readiness of the device and also the device operator is free to monitor the device when it is not being used on a patient and so they can attend to any failure or maintenance without being hindered by having to deal with an emergency situation.

According to yet a further aspect of the invention, there is provided a method of self testing a medical device said medical device being arranged to transmit information concerning components of said medical device to an indicator, wherein one or more components are caused to carry out a self test, the results of the self test are stored and on operation of said medical device are transmitted to a processor for analysis and display by said indicator. Preferably, the self test is a periodic or a aperiodic self test, activated by a timing device. It is envisaged information concerning said components is sent to a summator prior to being sent to a processor.

An embodiment of the invention will now be described, by way of example only, with reference to the accompanying figures in which:

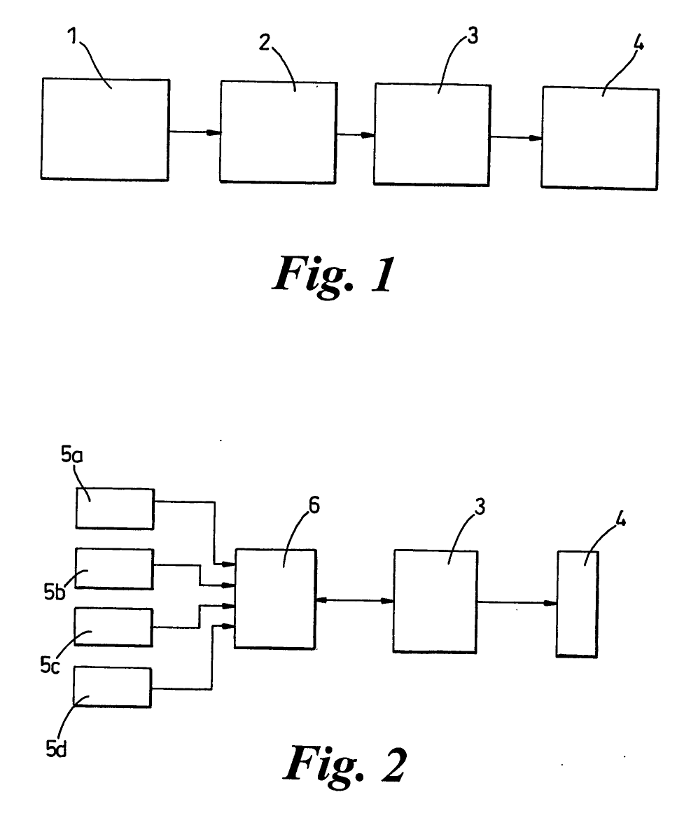

FIG. 1 shows a block diagram of an existing self testing component system,

FIG. 2 shows a block diagram of self testing components according to an embodiment of the invention,

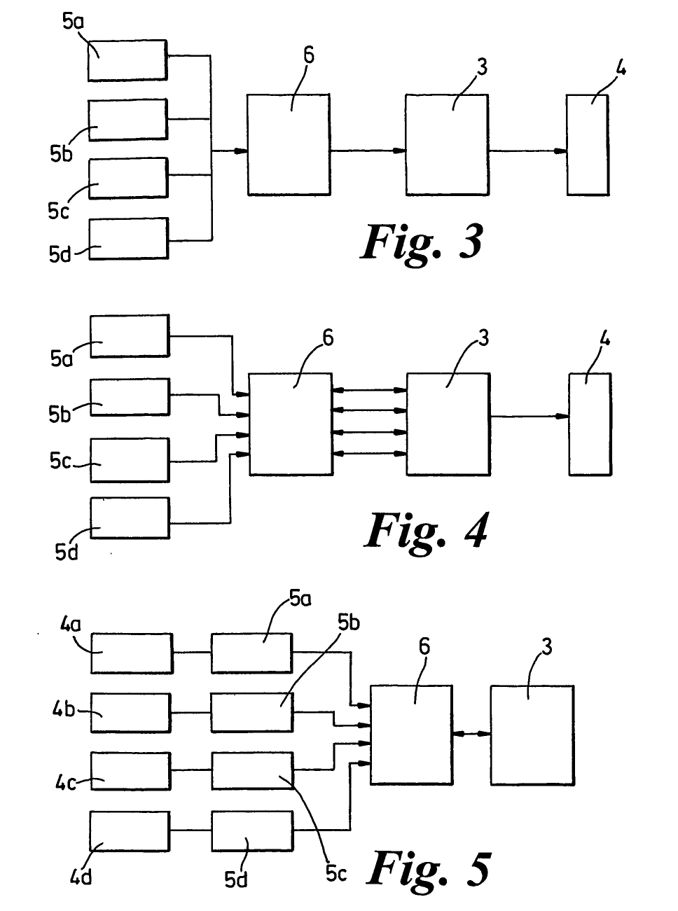

FIG. 3 is an alternative arrangement of self testing components to that of FIG. 2,

FIG. 4 shows yet another arrangement of self testing components of an embodiment of the invention,

FIG. 5 shows a block diagram of components of the invention where separate indicators for the components being tested for the system is shown,

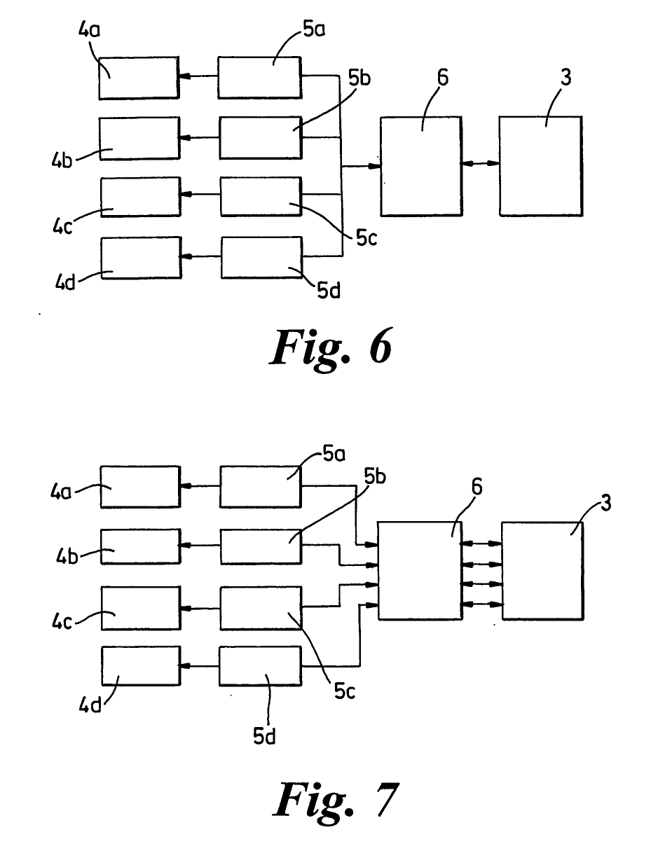

FIG. 6 shows yet another arrangement of components and indicators of a self test arrangement,

FIG. 7 shows a further arrangement of components according to an embodiment of the invention,

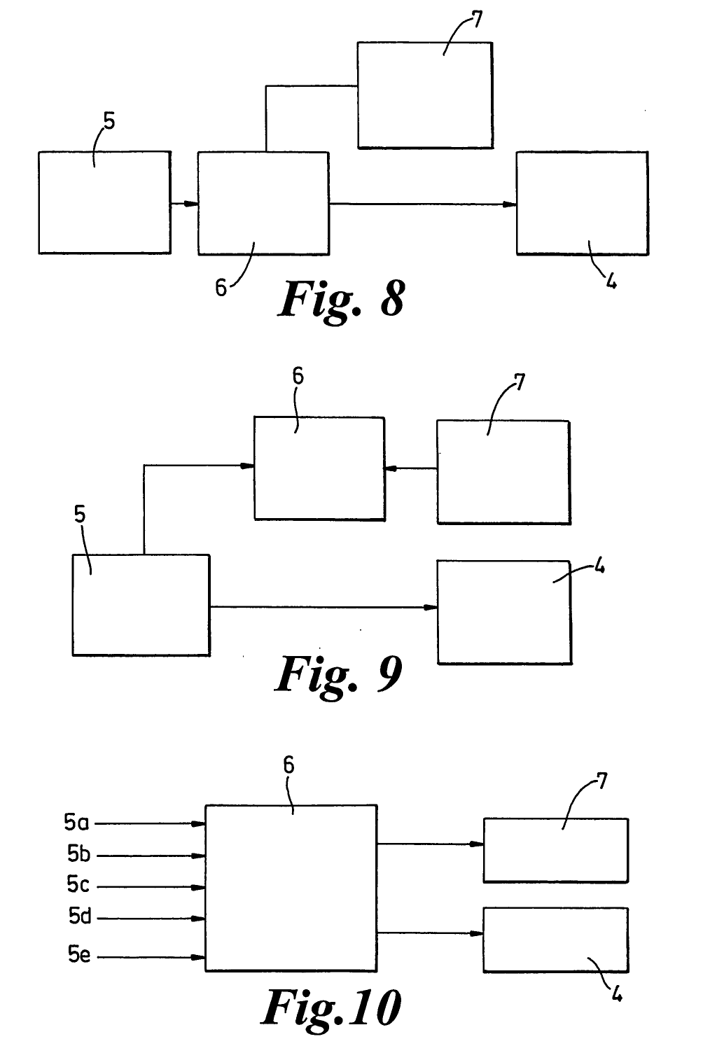

FIG. 8 shows a block diagram of components where the indicator is in direct contact with a summator,

FIG. 9 shows an arrangement where a summator process works in tandem to check the results of self testing of components,

FIG. 10 shows a block diagram representation of self testing apparatus using separate inputs to a summator and two outputs, one being to a processor, and the other to an indicator.

As shown in FIG. 1, known self test systems are relatively simple where a test signal generated from a power source at 1, relays a signal to a processor which causes testing of the components using a self test circuit at 2. The results of the testing of the component are fed to a processor 3 which collates data about the status of the component and further data from that component, such as whether the impedance or current flow through the component falls within or outside certain parameters. The results of the test, for example whether the data collated about the component are within defined limits or outside defined limits are displayed by an indicator 4. The indicator can either display defined values or give an indication whether the data falls within a certain range.

As shown in FIG. 2, components such as an electrode pad, circuitry, addition of the communication system of a medical device, the modem and the battery level, examples of such components being shown by references 5a to 5d, each are connected to test circuitry and each of the components has a dedicated communication link to a summator 6. A typical summator that could be used is a type 112-60 summator which is designed to sum together digital pulse signals. Such a device can act as an adder/subtractor and typically two outputs are provided for use with an external add/subtract counter. The summator can be matched to the amplitude, frequency and pulse of each input signal and receive, for example, 24 volts pulse outputs. Typically, the circuit consists of up to five identical isolated input stages. Input signals pass through these stages and are temporarily stored in five latches. The stored pulses are scanned out sequentially to provide the summed output pulses when the summator is operating in the adder version. The design ensures that coincidental or overlapping input pulses are accepted. An adder/subtractor version permits the use of add/subtract counters which cannot accept coincident add and subtract pulses. A typical permitted input frequency for a summator would depend on the number of inputs used and the required output pulse. For example, for an electromagnetic counter output (60 ms pulse) an output rate of 10 Hz maximum is permitted. This would give a maximum of 1 Hz for each of 5 inputs, or 2.5-Hz for two outputs. Typically the relationship-between input and output is given by the following formula:
Output=maximum input rate×n×2 where n a number of input channels.

Higher input frequencies, giving a maximum amount per pulse rate of up to 5 KHz (for example two inputs at 1.25 KHz or 5 outputs at 0.5 KHz) are also available.

The components 5(a) to 5(d) can carry out a self test routine, independently of the processor. Self testing is by way of measurement, for example, of impedance or voltage across electrodes that are integral with a component. The summator 6 provides values for the components 5(a) to 5(d) and latches the results, and these latched results can be fed to a processor that collates the results from the summator. The processor includes processing circuitry and preferably an internal memory to generate output signals. In the processor, the summated data is collated with data held for the required parameters of condition for the various components that are being self tested and the data can then be collated to produce a code that is communicated to an indicator 4.

As shown in FIG. 3, data from the individual components 5(a) to 5(d) is fed as an output from test circuits for the individual components and these are fed to a single communication output as a collated signal, which passes to the summator 6. Information from the summator is then fed by a single communication link to a processor 3 and again the central processing unit in the processor collates the information from the summator with stored memory data concerning the components being tested. As shown, a single communication link sends an output to the indicator 4, for example a digital display, to show the status of the combined data from the components 5(a) to 5(d). The indicator 4 can then give a yes or no indication of whether the entire number of components for the medical device are in operable condition or alternatively, an indication may be given that one or more components are either to be self tested or are not in a fit condition for use.

FIG. 4 shows an alternative arrangement to that shown in FIG. 3 where individual signals are sent from self test circuits for individual components, and these are fed to the summator 6 by individual communication links. Data from the components will then be sent to the summator 6 where individual outputs for various components are fed through the summator to the processor about the status of the components 5a to 5d. The processor 6 can then collate this information and if required there can be a reverse link back to the summator so that a second test can be carried out for the components. This routine between the summator and the processor can be carried out over a set period or a set number of times to check whether the data from the summator complies with data held in the processor and after this set routine has been carried out, an output signal from the processor can be transmitted to the indicator to display whether or not the individual components or the components as a whole have been self tested and comply with parameters that would allow the medical device to be used.

FIG. 5 shows an alternative arrangement where individual components 5a to 5d are tested using a self test circuit. Individual lines from components 5a to 5d are fed to a summator 6 which communicates with processor 3 by way of a single combined link which can check whether the summated data from the components complies with those parameters and information stored in the processor at 3. The data concerning the individual components can then be displayed on individual displays for each component at displays 4a to 4d. This would give an independent indication for each component as to whether it is in a satisfactory condition for use. However, if an individual component is found not to be suitable for use, an indication would be given on the display for that component.

FIG. 6 shows an alternative arrangement to that shown in FIG. 5 where rather than having individual outputs from circuits associated with components 5a to 5d, the outputs from each test circuit for the component is combined into a signal input to summator 6. Summator 6 communicates with processor 3 to verify information concerning the components and information concerning the components is displayed on individual displays for each component 4a to 4d.

FIG. 7 is an alternative embodiment of the arrangement shown in FIG. 6 where instead of signals from the test circuit for the individual components 5a to 5d being combined to form a single signal to the processor 3, the signals from the test circuits for the components 5a to 5d are kept as individual communication signals which form separate inputs to the summator 6. Separate outputs for each component are transmitted from the summator and are sent to the processor which can operate a number of test routines by communicating over and over again for either a given time period or for a set number of test routines with the summator to check that the information from the summator is consistent and once this test routine has been carried out, individual signals will be sent to individual indicators 4a to 4d about separate components.

In the case of FIG. 8, the components are self tested independently. The results are fed to a summator 6 and latched until the next test result for a component is received. The test may be for an individual component that has been tested a set number of times or over a set-time period to achieve a single set of results for that component with the results being summated. Alternatively, results for different components can be fed to the summator and latched until a set number of test results for separate components is achieved. The latched results are then sent to a processor 3, which conducts a self test upon response from an input from the summator. The processor can operate as a result of a response from a single signal from a test button or upon receiving a signal from the base charger from a server controlling the test apparatus. In this case, the operator of the self test can check the self test status using the indicator 4 or alternatively the processor looks at the summator self status. This arrangement allows the indicator to be operated independently of the processor and directly from the summator.

As shown in FIG. 9, the indicator 4 may be directly linked to the self test facility for the components 5. The self test facility can be provided as a processor that produces for example a signal. A typical processor will be a 555 timer having a clock counter, which will provide about 60 pulses per minute to, for example pads. A current drop through the pads equals impedance and the processor in the pad can measure the impedance. A signal is then generated which is then fed to the summator and latched until the next result from a measurement of the impedance is passed to the summator 6. The processor then calculates the values of a series of results from the summator or a combined result from the summator to see if it falls within certain values for the component and the whole result can then be indicated to display whether the component is in a condition to operate.

FIG. 10 shows a particular embodiment where separate input lines for in this case five components are fed to the summator and two output lines are fed through the summator, one to the processor 6 and one to the indicator 4. Information from the summator then passes to the to the indicator for display.

Although a preferred embodiment of the invention has been described, it will be understood by those skilled in the art that the invention can be performed in various ways without departing from the true spirit and scope of the invention.

1-32. (canceled)

33. A self test system for a medical device comprising a plurality of components each of which has a respective self-test means associated therewith capable of carrying out a self-test routine on the associated component, wherein each of said self-test means is operable to be activated independently of operation of the medical device and not by a signal from a processor associated with said medical device, with the results thereof being passed to a common processor.

34. A self test system for a medical device according to claim 33, wherein the self test is activated independently of operation of the medical device and not by a signal from the centralized processor associated with said medical device.

35. A self test system for a medical device according to claim 33, wherein the self test system includes a summator which receives data from the one or more self test units about said components, the summator storing said data so that it can be transmitted to an indicator either directly or via a processor which can access said data.

36. A self test system for a medical device said medical device being arranged to transmit information concerning components of said medical device to an indicator which can show the status of the components when tested, characterised in that the self test system comprises one or more self test units which can self test one or more individual components of the medical device and a summator which receives data from the one or more self test units about said components, the summator storing said data so that it can be transmitted to an indicator either directly or via a processor which can access said data.

37. A self test system according to claim 33, wherein the one or more components each has a dedicated self test unit.

38. A self test system according to claim 33, wherein a plurality of the one or more components communicate with a self test unit.

39. A self test system according to claim 33, wherein the data from the self test is fed by a single data link to the summator.

40. A self test system according to claim 33, wherein the data from the self test units is fed by a plurality of separate data links to the summator.

41. A self test system according to claim 34, wherein the summator is a separate counter/adder component, or a micro-controller.

42. A self test system according to claim 34, wherein the summator is or includes a subtractor component.

43. A self test system according to claim 34, wherein the summator is part of a main microprocessor.

44. A self test system according to claim 34, wherein data from the, or each component is delivered to the summator as a signal comprising a number of pulses.

45. A self test system according to claim 44, wherein the pulses are identified as discrete numbers of pulses to the value of x² or as a prime number.

46. A self test system according to claim 44, wherein the number of pulses for components making up an AED are any of the following:

| | |
|---|---|
| Electrode condition | 32² = 1024 pulses |
| Battery on charge | 33² = 1089 pulses |
| System condition | 34² = 1156 pulses |
| Modem condition | 35² = 1225 pulses |

47. A self test system according to claim 33, wherein a self test for a component is triggered by a test having been carried out on another component.

48. A self test system according to claim 33, wherein the test is activated by digital signal processor.

49. A self test system according to claim 48, wherein the digital signal processor is activated by a signal from a server or base station in contact with the medical device.

50. A self test system according to claim 33, wherein the self test is activated by the medical device being placed in a base station for said device.

51. A self test system according to claim 33, wherein a self test involves testing the voltage across substantially all of the circuitry of the medical device.

52. A self test system according to claim 48, wherein the test is carried out either at a first voltage of between 450V or a second voltage of 40V.

53. A self test system according to claim 33, wherein the indicator is a digital display that can display results for the summator or processor independently of one another.

54. A self test system according to claim 53, wherein the processor reviews the results of the summator prior to the results being fed to the indicator.

55. A self test system according to claim 53, wherein the number of pulses being fed to the summator is recorded.

56. A self test system according to claim 55, wherein the number of pulses is measured against set parameters to provide an indication of whether one or more components are functioning as required.

57. A self test system according to claim 33, wherein the self test system tests the condition of the power

58. A self test system according to claim 33, wherein the testing of components is based on testing a sample of signals over time or testing a defined number of signals for each component.

59. A self test system according to claim 33, wherein the indicator is integral with said medical device.

60. A self test system according to claim 33, wherein the indicator is a separate component associated with said medical device.

61. A method of self testing a medical device said medical device being arranged to transmit information concerning components of said medical device to an indicator, wherein one or more components are caused to carry out a self test, the results of the self test are stored and on operation of said medical device are transmitted to a processor for analysis and display by said indicator.

62. A method according to claim 61, wherein the self test is a periodic or aperiodic self test, activated by a timing device.

63. A method according to claim 61, wherein information concerning said components is sent to a summator prior to being sent to a processor.
